(19) United States
(12) Patent Application Publication
(10) Pub. No.: US 2025/0122128 A1
(43) Pub. Date: Apr. 17, 2025

(54) Preparation Method of Rice Straw Biochar Loaded with Bacillus cereus and Use Thereof

(71) Applicants: Sichuan Agricultural University, Yaan City (CN); China National Tobacco Corporation Sichuan Company, Chengdu City (CN); Sichuan Provincial Tobacco Company Liangshan Prefecture Company, Xichang City (CN)

(72) Inventors: Bing Li, Yaan City (CN); Jie Li, Yaan City (CN); Changchun Feng, Yaan City (CN); Changquan Wang, Yaan City (CN); Rong Huang, Yaan City (CN); Yulan Chen, Yaan City (CN); Qi Tao, Yaan City (CN)

(21) Appl. No.: 18/665,135

(22) Filed: May 15, 2024

(30) Foreign Application Priority Data
Oct. 17, 2023 (CN) 202311339324.3

(51) Int. Cl. C05F 11/08; C05D 9/00; C12N 1/20; C12R 1/085

(52) U.S. Cl. CPC C05F 11/08; C05D 9/00; C12N 1/205; C12R 2001/085

(57) ABSTRACT

A preparation method of a rice straw biochar loaded with Bacillus cereus, comprising: (1) washing, drying, grinding and sieving rice straws; (2) performing anaerobic pyrolysis treatment on the rice straws treated in the step (1) at 300-700° C. to obtain a biochar; (3) treating the biochar obtained in the step (2) with hydrochloric acid, then washing until a washing solution is neutral, drying, grinding, sieving and sterilizing to obtain a sterilized biochar; and (4) performing mixed culture on the sterilized biochar and a solution of Bacillus cereus subjected to activation culture, and then centrifuging after the end of culture to remove supernatant, so as to obtain a rice straw biochar loaded with Bacillus cereus having a carbon immobilizing capability.

### CROSS REFERENCE TO RELATED APPLICATION

This application claims the benefit of and takes priority from Chinese Patent Application No. 202311339324.3 filed on Oct. 17, 2023, the contents of which are herein incorporated by reference.

### TECHNICAL FIELD

The present disclosure belongs to the technical field of soil improvement, and particularly relates to a method for increasing the content of soil organic carbon.

### BACKGROUND

As the largest carbon reservoir in a terrestrial biosphere, soil serves as source and sinks in a global carbon cycle. Organic carbon as a component in the soil carbon reservoir has up to 1500 Pg in content. Organic carbon plays an important role in maintaining soil fertility, increasing crop yields, improving climate conditions and the like. Meanwhile, the activities of soil organisms and the growth and death of plants have important significance in the aspects of promoting soil organic carbon cycle and mitigating greenhouse gas effects. Therefore, maintaining the stability of soil organic carbon and increasing the content of organic carbon in impoverished soil have great strategic significance in sustainable agricultural production.

In the traditional agricultural farming, improper farming measures such as straw burning and tillage have gained recognition in the aspects of causing the decrease in soil organic carbon content, greenhouse gas emission, air pollution and the like. Therefore, protective farming manners, application of organic fertilizers, straw returning to the field and other measures are proposed to increase the content of soil organic carbon and improve the crop productivity; however, these measures are highly controversial in terms of leading to greenhouse gas emissions.

In recent years, the biochar obtained by pyrolysis of agricultural wastes such as crop straws, livestock manure and rice husks under oxygen limiting/anaerobic and high temperature conditions is a soil improver, which is widely evaluated in the aspects of increasing the content of soil organic carbon, increasing the yield of crops, reducing greenhouse gas emissions and the like. The nature of the biochar in improving the content of soil organic carbon is that the content of soil organic carbon is increased by changing the abundance and metabolic activity of soil organisms. There are microorganisms that immobilize CO₂ and are transformed into organic matters in soil, and the storage rate of soil organic carbon may be increased by improving the abundance of carbon immobilizing organisms. Further, the application of the biochar loaded with carbon immobilizing bacteria into soil may achieve a dual-effect carbon enhancement function of biochar and carbon immobilizing bacteria.

### SUMMARY

The technical problem to be solved by the present disclosure is to provide a preparation method of a rice straw biochar loaded with strains having a carbon immobilizing capability, such that the biochar and carbon immobilizing bacteria increase the content of soil organic carbon and enhance the stability of soil organic carbon in dual effects.

The technical solution of the present disclosure is as follows: a preparation method of a rice straw biochar loaded with Bacillus cereus having a carbon immobilizing capability, comprising the following steps:
(1) washing, drying, grinding and sieving rice straws;
(2) performing anaerobic pyrolysis treatment on the rice straws treated in the step (1) at 300-700° C. to obtain a biochar;
(3) treating the biochar obtained in the step (2) with hydrochloric acid, then washing until a washing solution is neutral, drying, grinding, sieving and sterilizing to obtain a sterilized biochar; and
(4) performing mixed culture on the sterilized biochar obtained in the step (3) and a Bacillus cereus liquid subjected to activation culture, and then centrifuging after the end of culture to remove supernatant, so as to obtain a rice straw biochar loaded with Bacillus cereus, the Bacillus cereus being Bacillus cereus deposited under accession No.: CGMCC 1.15914.

Further, in the step (1), the sieving refers to passing through a 20-mesh sieve.

Further, in the step (2), the anaerobic pyrolysis refers to pyrolysis under the protection of nitrogen.

Further, in the step (2), the program of anaerobic pyrolysis is as follows: the temperature is raised to 300-700° C., preferably 500° C., at a rate of 10° C./min and then maintained for 2 h.

Further, in the step (3), a hydrochloric acid treatment method is as follows: the biochar is soaked for 4 h using 0.5 mol/L hydrochloric acid, and the sieving refers to passing through a 60-mesh sieve.

Further, in the step (4), a mixed ratio of Bacillus cereus liquid to sterilized biochar is 1 g:10 ml; and the culture time is 24 h.

Further, in the step (4), the OD₆₀₀ value of the Bacillus cereus is 1.

Provided is use of the rice straw biochar obtained by the above-mentioned preparation method in increasing the content of soil organic carbon, achieving the stable input of Bacillus cereus into soil or enhancing the stability of soil organic carbon.

Compared with the prior art, the present disclosure has the following beneficial effects:
the rice straw biochar loaded with Bacillus cereus having a carbon immobilizing capability prepared by the method of the present disclosure is adopted, a material is composed of rice straws and Bacillus cereus, that is, raw straws are prepared into biochar under the anaerobic conditions, and the improved biochar is loaded with Bacillus cereus, thereby achieving the proliferation of Bacillus cereus on biochar and increase of the contents of the organic carbon components in biochar. If applied to soil, the rice straw biochar loaded with Bacillus cereus can increase the content of soil organic carbon.

### BRIEF DESCRIPTION OF THE DRAWINGS

FIG. 1 is a viable count chart of Bacillus cereus in example 1.

FIG. 2 is a dissolved organic carbon content graph of a biochar in example 1.

FIG. 3 is an easily oxidizable carbon content graph of a biochar in example 1.

FIG. 4 is a microbial biomass organic carbon content graph of a biochar in example 1.

FIG. 5 is a dissolved organic carbon content graph of a biochar in example 2.

FIG. 6 is a soil mineral bound organic carbon graph of a biochar in example 2.

FIG. 7 is an absolute abundance chart of soil Bacillus genus in example 2; wherein absolute abundance is represented by 1 g (absolute abundance×100); different lowercase letters represent significant difference (p<0.05).

### DETAILED DESCRIPTION OF THE EMBODIMENTS

Experimental methods in examples below, unless specified stated, are all conventional methods. Experimental materials used in examples below, unless specified stated, are all commercially available.

Bacillus cereus used in the present disclosure is commercially available from China General Microbiological Culture Collection Center under accession No.: CGMCC1.15914.

### Example 1 Study on Proliferation of Bacillus cereus on Rice Straw Biochar and Contents of Organic Carbon Components in Biochar

### 1. Specific Steps

### 1.1 Preparation of Bacteria-Loaded Biochar

Rice straws were washed, dried, grinded and passed through a 20-mesh sieve and then put in a muffle furnace, nitrogen was introduced in the muffle furnace, the above rice straws were heated to 300° C., 500° C. and 700° C. at a heating rate of 10° C./min, and the temperatures were maintained for 2 h, so as to obtain a 300° C. biochar, a 500° C. biochar and a 700° C. biochar. The above biochars were treated with 0.5 mol/L hydrochloric acid for 4 h, and the biochars were treated in hydrochloric acid to reduce hazardous substances. Subsequently, the treated biochars were washed until washing solutions were neutral. The washed biochars were dried, sieved, passed through the sieve and sterilized in sequence to obtain a 300° C. sterilized biochar, a 500° C. sterilized biochar and a 700° C. sterilized biochar. Bacillus cereus was cultured by using a glucose asparagine agar culture medium, the strains and sterilized biochars were mixed and cultured for 24 h separately in a ratio of 10 ml of bacterial liquid to each g of sterilized biochar after the OD₆₀₀ value of the strains was adjusted to 1, and a 300° C. bacteria-loaded biochar, a 500° C. bacteria-loaded biochar and a 700° C. bacteria-loaded biochar were obtained after centrifuging and removing supernatant.

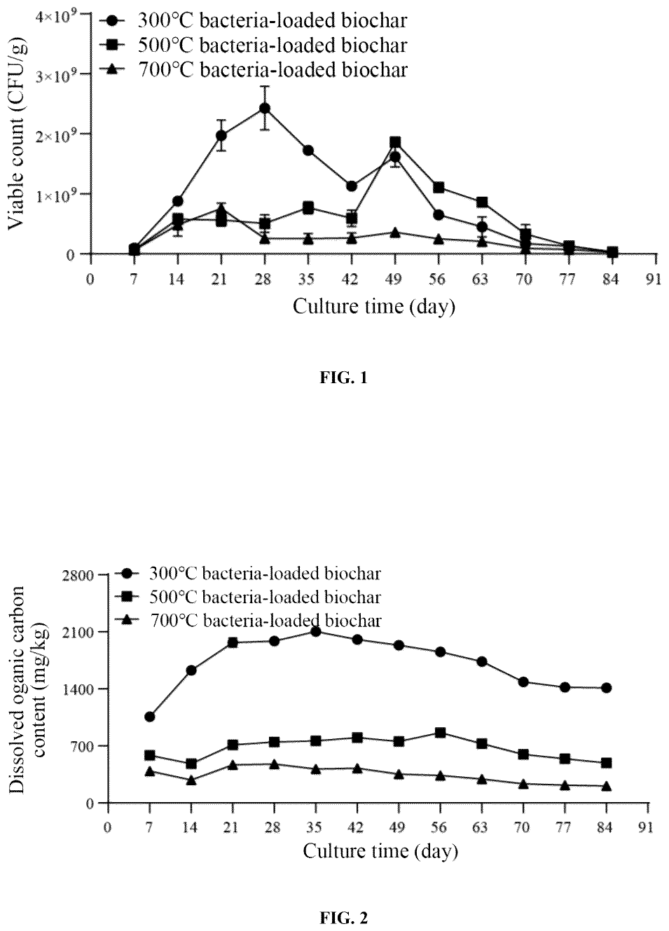

As shown in FIG. 1, as the preparation temperature rises, the specific surface area, total pore volume, micropore volume and mesopore volume of the biochar increase, which are maximally 262.68 m²/g, 0.16 cm³/g, 0.07 cm³/g and 0.08 cm³/g respectively; the average pore diameter, dissolved organic carbon and easily oxidizable organic carbon decrease with the preparation temperature. The appropriate physical properties and organic carbon components of the biochar provide conditions for colonization and growth of microorganisms.

TABLE 1
Properties of biochars prepared at different temperatures

| | | | | | | | Easily |
|---|---|---|---|---|---|---|---|
| | Specific | Average | Total | Micro- | Meso- | Dissolved | oxidizable |
| | surface | pore | pore | pore | pore | organic | organic |
| | area | diameter | volume | volume | volume | carbon | carbon |
| Biochar | (m²/g) | (nm) | (cm³/g) | (cm³/g) | (cm³/g) | (mg/kg) | (mg/kg) |
| 300° C. | 43.33 | 4.05 | 0.04 | 0.03 | 0.01 | 1421.43 ± 75.51 | 141.71 ± 8.48 |
| biochar | | | | | | | |
| 500° C. | 116.01 | 3.12 | 0.09 | 0.06 | 0.03 | 424.34 ± 41.01 | 86.31 ± 4.22 |
| Biochar | | | | | | | |
| 700° C. | 262.68 | 2.41 | 0.16 | 0.07 | 0.08 | 129.94 ± 20.27 | 80.85 ± 11.73 |
| Biochar | | | | | | | |

### 1.2 Culture of Bacteria-Loaded Biochar

After a bacteria-loaded biochar was dried in air on a clean bench, 35 g of bacteria-loaded biochar was cultured in a glass tissue culture bottle for 84 days at the culture temperature of 28° C., wherein the culture moisture content was 30% (moisture content based on weight). During the culture, a differential weight method was used to replenish the lost moisture.

### 1.3 Determination of Viable Counts

Viable counts of Bacillus cereus on a 300° C. biochar, a 500° C. biochar and a 700° C. biochar were determined by using a viable counting method.

### 1.4 Determination on Contents of Organic Carbon Components on Biochar

Dissolved organic carbon on a sample was taken by shaking with deionized water in a ratio of 1:20, and analyzed by using a total organic carbon (TOC) analyzer after an extracting solution passed through a 0.45 μm filtration membrane. The content of easily oxidizable organic carbon was determined by using KMnO₄, and the content of microbial biomass organic carbon was determined by using a CHCl₃ fumigation-K₂SO₄ extraction method.

### 2. Experimental Results

2.1 Viable Counts of Bacillus cereus

It can be seen from results in FIG. 1 that the total viable count of the 300° C. bacteria-loaded biochar reaches a peak value which is 2.43×10⁹ CFU/g on day 28; the peak value of the total viable count of the 500° C. bacteria-loaded biochar has a lag effect, which appears on day 49 and is 1.86×10⁹ CFU/g; the total viable count of the 700° C. bacteria-loaded biocha reaches a peak value which is 7.55×10⁸ CFU/g on day 21. The results show that Bacillus cereus can grow on biochars prepared at different temperatures. At the later stage of growth, Bacillus cereus immobilizes CO₂ to achieve sustained proliferation, and the strains have better exhibition on the 500° C. biochar.

### 2.2 Change in Organic Carbon Components on Bacteria-Loaded Biochars at Different Temperatures

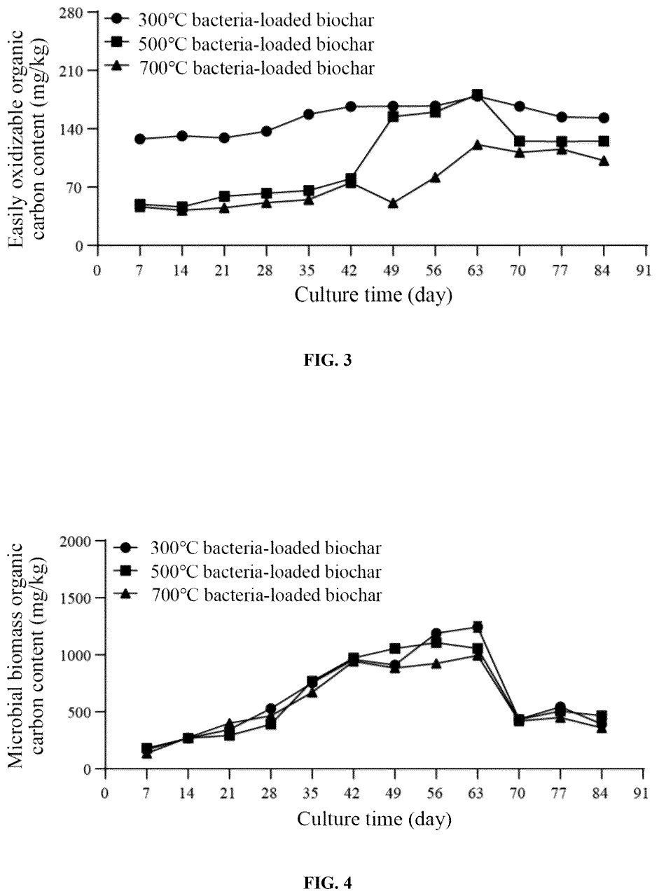

It can be seen from results in FIG. 2, FIG. 3 and FIG. 4 that the contents of dissolved organic carbon, easily oxidizable organic carbon and microbial biomass organic carbon are increased compared with background values, showing a trend of first rising and then falling. The contents of dissolved organic carbon reach peak values on day 35, day 56 and day 28, which are 2104.19 mg/kg, 862.53 mg/kg and 477.52 mg/kg respectively. The contents of easily oxidizable organic carbon are lower than the background values in the early stage and in the middle stage. The contents of easily oxidizable organic carbon in three groups reach peak values on day 63, which are 179.01 mg/kg, 181.07 mg/kg and 120.90 mg/kg respectively. The contents of microbial biomass organic carbon in three groups reach peak values on day 63, day 56 and day 63 respectively, which are 1244.91 mg/kg, 1105.11 mg/kg and 994.36 mg/kg respectively. The results show that Bacillus cereus is proliferated by utilizing dissolved organic carbon and easily oxidizable organic carbon in the early and middle stages; the biochar immobilizes CO₂ in the later stage to increase the contents of organic carbon components in the biochar, so the biochar has a carbon immobilizing capability. Where, the contents of three organic carbon components in the 500° C. biochar reach maximum values in the later stage, showing that Bacillus cereus has a better CO₂ utilizing capacity on the 500° C. biochar.

### Example 2 Comparison Study on Increase in Contents of Soil Organic Carbon by Biochar and Bacteria-Loaded Biochar

### 1. Specific Steps

### 1.1 Experimental Design

The preparation of a biochar and a bacteria-loaded biochar is as shown in the preparation of a bacteria-loaded biochar described in 1.1 of example 1. The prepared biochar and bacteria-loaded biochar were respectively mixed with calcareous purple soil developed from residual slope deposits weathered by Jurassic Penglaizhen Formation brownish purple sandstone and mudstone in a ratio of 1% and then cultured in a glass tissue culture bottle, meanwhile, a control group was set. The culture temperature was 28° C., the culture moisture content was 30% (moisture content based on weight), and during the culture, a differential weight method was used to replenish the lost moisture, with culture time of 63 days.

### 1.2 Analysis on Soil Organic Carbon Components

A potassium dichromate-concentrated sulfuric acid plus heating method was used to determine the content of soil organic carbon, and (NaPO₃)₆ was used to determine the content of mineral bound organic carbon.

### 1.3 Microbial Diversity Analysis

Total DNA was extracted using a soil DNA rapid extraction kit. A primer pair, namely 338F:5′-ACTCCTACGGGAGGCAGCA-3′ and 806R:5′-GGACTACHVGGGTWTCTAAT-3′, was used to amplify V3-V4 regions of a bacterial 16s rRNA gene. Sequencing and analysis of amplifiers were performed on Illumina NovaSeq platform.

### 2. Experimental Results

### 2.1 the Contents of Soil Organic Carbon Components

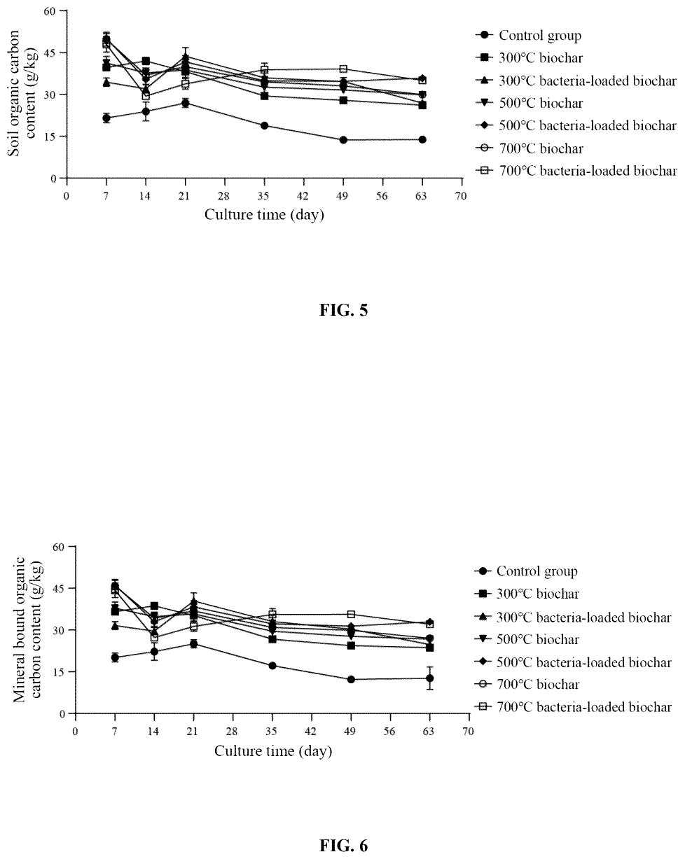

It can be seen from results in FIG. 5 and FIG. 6 that the addition of the biochar and bacteria-loaded biochar can significantly increase the contents of soil organic carbon and mineral bound soil organic carbon, and their increases have consistency. The bacteria-loaded biochar can more significantly increase the content of soil organic carbon by 36.38%-136.34%. The contents of soil organic carbon and soil mineral bound organic carbon in 500° C. bacteria-loaded biochar group on day 63 are both higher than those in other groups, which are 35.83 g/kg and 32.90 g/kg respectively, showing that the 500° C. bacteria-loaded biochar has better sustainability in the aspect of increasing the content of soil organic carbon. The results show that the bacteria-loaded biochar mainly increases the content of soil mineral bound organic carbon, which is beneficial for maintaining the stability of soil organic carbon.

### 2.2 Abundance Analysis of Bacillus Genus

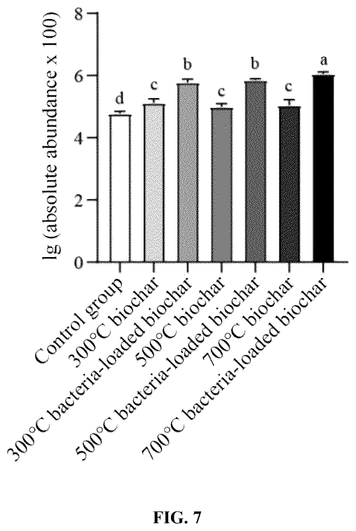

It can be seen from results in FIG. 7 that the addition of the biochar and bacteria-loaded biochar both can increase the absolute abundance of Bacillus genus, the absolute abundance of the bacteria-loaded biochar is obviously increased, and the absolute abundances of the Bacillus genus of the bacteria-loaded biochar are 5.78, 5.85 and 6.05 respectively. The results show that Bacillus genus can be mediated by the biochar and proliferated in soil. Therefore, the application of the biochar loaded with carbon immobilizing bacteria into soil can achieve the dual-effect carbon enhancement function of the biochar and the carbon immobilizing bacteria.

What is claimed is:

1. A preparation method of a rice straw biochar loaded with Bacillus cereus having a carbon immobilizing capability, comprising the following steps:
(1) washing, drying, grinding and sieving rice straws;
(2) performing anaerobic pyrolysis treatment on the rice straws treated in the step (1) at 300-700° C. to obtain a biochar;
(3) treating the biochar obtained in the step (2) with hydrochloric acid, then washing until a washing solution is neutral, drying, grinding, sieving and sterilizing to obtain a sterilized biochar; and
(4) performing mixed culture on the sterilized biochar obtained in the step (3) and a Bacillus cereus liquid subjected to activation culture, and then centrifuging after the end of culture to remove supernatant, so as to obtain a rice straw biochar loaded with Bacillus cereus, the Bacillus cereus being Bacillus cereus deposited under accession No.: CGMCC 1.15914.

2. The preparation method according to claim 1, wherein in the step (1), the sieving refers to passing through a 20-mesh sieve.

3. The preparation method according to claim 1, wherein in the step (2), the anaerobic pyrolysis refers to pyrolysis under the protection of nitrogen.

4. The preparation method according to claim 1, wherein in the step (2), the program of anaerobic pyrolysis is as follows: the temperature is raised to 300, 500 or 700° C. at a rate of 10° C./min and then maintained for 2 h.

5. The preparation method according to claim 1, wherein in the step (3), a hydrochloric acid treatment method is as follows: the biochar is soaked for 4 h using 0.5 mol/L hydrochloric acid, and the sieving refers to passing through a 60-mesh sieve.

6. The preparation method according to claim 1, wherein in the step (4), a mixed ratio of a Bacillus cereus liquid to sterilized biochar is 1 g:10 ml; and the culture time is 24 h.

7. The preparation method according to claim 6, wherein in the step (4), the OD₆₀₀ value of the Bacillus cereus liquid is 1.

8. Use of the rice straw biochar obtained by the preparation method according to claim 1 in increasing the content of soil organic carbon, achieving the stable input of Bacillus cereus into soil, or enhancing the stability of soil organic carbon.
